(19) United States
(12) Patent Application Publication
(10) Pub. No.: US 2018/0228856 A1
(43) Pub. Date: Aug. 16, 2018

(54) Antibacterial combination of Basil oil and Taiwan red cypress oil for preventing drug-resistant S. aureus infections and the use thereof

(71) Applicant: Chiuan Ho Biomedical Co.,LTD, Hsinchu City (TW)

(72) Inventor: Cheng Hung Huei, Hsinchu City (TW)

(21) Appl. No.: 15/430,945

(22) Filed: Feb. 13, 2017

(51) Int. Cl. A61K 36/53; A61K 36/14

(52) U.S. Cl. CPC A61K 36/53; A61K 2236/331; A61K 36/14

(57) ABSTRACT

The present invention relates to a combination of Basil oil and Taiwan red cypress oil exhibiting synergistic antimicrobial activity. The present combinations comprise two essential oils, leaves of Basil (Ocimum basilicum) and heartwood of Taiwan red cypress (Chamaecyparis formosensis Matsum.), at a ratio of 1:2 to 2:1. The antibacterial combinations can effectively prevent drug resistant S. aureus infections, especially the Methicillin-resistant Staphylococcus aureus (MRSA) infections.

### BACKGROUND OF THE INVENTION

### Technical Field of the Invention

the present invention relates to an antibacterial combination of essential oils used for preventing drug resistant S. aureus infections. Especially, the present invention relates to a synergic combination of Basil oil and Taiwan red cypress oil for preventing Methicillin-resistant Staphylococcus aureus (MRSA) infections.

### Background

In Gram-positive bacteria, Staphylococcus aureus is the most important and common pathogen in clinical medicine. It can cause a wide spectrum of diseases ranging from mild skin and soft tissue infections to severe life threatening pneumonia, bacteremia, endocarditis, and osteomyelitis (Lowy, N Engl J Med 339(8): 520-532, 1998). Methicillin, a semi-synthetic penicillin refractory to penicillinase, was introduced in 1959, but methicillin-resistant S. aureus (MRSA) was found soon after (Jevons, Br Med J 1: 124-125, 1961). The prevalence of MRSA steadily increased in 1980s to 1990s to become the leading cause of hospital-associated infections (HA-MRSA). In the late 1990s, community-associated MRSA (CA-MRSA) also emerged worldwide in patients without any healthcare associated risk factors (David and Daum, Clin Microbiol Rev 23(3): 616-687, 2010).

In addition to being resistant to most of currently available β-lactams in the market, MRSA strains are usually resistant to several other classes of non-β-lactams. Vancomycin has been the last line of defense against serious MRSA infections. Over the past decade, the minimum concentration of vancomycin required to inhibit MRSA has been gradually increasing, suggesting that the susceptibility to vancomycin is decreasing. The emergence of vancomycin intermediate and resistant S. aureus (VISA and VRSA) further compromises treatment options for multidrug resistant MRSA (Hiramatsu, Cui et al., Trends Microbiol 9(10): 486-493, 2001; Chang, Sievert et al., N Engl J Med 348(14): 1342-1347, 2003). Considering the inherent of S. aureus to rapidly develop antibiotic resistance, there is an urgent need for preventive strategies not only for the safety of patients, but also in terms of cost effectiveness on health care programs.

It has been shown that nasal carriage of S. aureus is a major risk factor for subsequent development of community-associated and nosocomial infections (von Eiff, Becker et al., N Engl. J Med. 344(1): 11-16, 2001) and eradication of S. aureus by intranasal application of mupirocin did significantly decrease the rate of all nosocomial S. aureus infections (Perl, Cullen et al., N Engl J Med 346(24): 1871-1877, 2002). The use of plant essential oils against MRSA has attracted great attention from many researchers (Doran, Morden et al., Lett Appl Microbiol 48(4): 387-392, 2009; Warnke, Becker et al., J Craniomaxillofac Surg 37(7): 392-397, 2009). In vitro studies suggest that some of essential oils and their vapors have strong bactericidal action. Essential oils are highly volatile at room temperature. The present invention firstly investigates the potential role of essential oils as inhalation eradication of MRSA carriage to prevent infections.

In the present invention, a combination of two essential oils, leaves of Basil (Ocimum basilicum) and heartwood of Taiwan red cypress (Chamaecyparis formosensis Matsum.), is found to have strong antibacterial activities against not only clinical methicillin-resistant (MRSA) strains but even a vancomycin intermediate (VISA) strain.

### SUMMARY OF INVENTION

Accordingly, in one aspect, the present invention relates to an antibacterial combination of Basil oil and Taiwan red cypress oil for preventing drug-resistant Staphylococcus aureus infections.

In some embodiments of the invention, the drug-resistant Staphylococcus aureus is a Methicillin-resistant Staphylococcus aureus (MRSA) strain. In other embodiments of the invention, the drug-resistant Staphylococcus aureus is a vancomycin intermediate (VISA) strain.

In some embodiments of the invention, the ratio (V/V) of Basil oil and Taiwan red cypress oil in the combination is in a range of 2:1 to 1:2. Preferably, the Basil oil and Taiwan red cypress oil are combined at a ratio of 1:1 (V/V).

In another aspect, the present invention relates to a method for inhibiting growth of drug-resistant Staphylococcus aureus, comprising a step of contacting the drug-resistant Staphylococcus aureus with an antibacterial combination of Basil oil and Taiwan red cypress oil.

In certain embodiments of the invention, the contacting step comprises applying the antibacterial combination to a surface containing the drug-resistant Staphylococcus aureus. In other embodiments of the invention, the contacting step comprises dispersing the antibacterial combination in an area containing the drug-resistant Staphylococcus aureus.

### BRIEF DESCRIPTION OF THE DRAWINGS

FIG. 1 shows the antibacterial activity of 13 essential oils against S. aureus, tested by direct contact using disk diffusion method. Zones of inhibition (mm) for essential oils against MRSA reference strain ATCC 43300 are presented in parentheses. Ethanol (75%) and sterile water were also included for serving as controls.

FIG. 2 shows the inhibition from the four extracted oils in the tested plate when tested in direct contact using disk diffusion method. The result of MRSA strain ATCC 43300 demonstrates the effect of combinations of essential oil with vapor activity. Escherichia coli ATCC 25922 was used as a control.

FIG. 3 shows the antibacterial activities of the four extracted oils against MRSA strain ATCC 43300. (A) Disk diffusion test and (B) vapor activity test were performed on the independent plate for each essential oil. Zones of inhibition (mm) are presented in parentheses.

FIG. 4 shows the vapor activity assay against MRSA strain ATCC 43300 in combinations of two essential oils from the four extracted oils.

FIG. 5 shows the vapor activity assay against MRSA strain ATCC 43300 by six effective commercial essential oils in combination with Basil oil or sterile water.

FIG. 6 shows the effect of vapor activity by the combination of Basil oil and Taiwan red cypress oil were tested against a daptomycin nonsusceptible VISA clinical strain Z172.

### DETAILED DESCRIPTION OF THE INVENTION

As use in herein, “essential oil” refers to a concentrated hydrophobic liquid containing volatile aroma compounds from plants. An oil is “essential” in the sense that it contains the “essence of” the plant's fragrance—the characteristic fragrance of the plant from which it is derived. Essential oils are generally extracted by distillation, often by using steam. Other processes include expression, solvent extraction, absolute oil extraction, resin tapping, and cold pressing.

The antibiotic combination according to the invention may be formulated for administration in any convenient way for use in human or veterinary medicine

The antibiotic oil formulation of the invention can be integrated in a soap product, a lotion product and a spray product. The inventive combination may be applied either directly or indirectly to the skin. Where a spray applicator is used, the solution comprising the inventive combination is sprayed onto each hand (or skin surface which is to be cleansed) and thereafter the skin is hand rubbed gently until it is dry.

The antibiotic essential oil combination of the invention can be formulated into a vaporizable composition. The vaporizable composition of essential oil may be dispersed in an area, such as a room, where a drug resistant S. aureus strain exists by an essential oil vaporizer. The vaporizer can be made in different materials, including ceramic, terra-cotta, metal and glass etc. The vaporizable composition can also be dispersed into a nasal cavity as simple as dropping the vaporizable antibiotic combination onto a tissue or handkerchief and smelling it.

### EXEMPLIFICATION

The other characteristics and advantages of the present invention will be further illustrated and described in the following examples. The examples described herein are using for illustrations, not for limitations of the invention.

### Example 1

### Preparation of Antibacterial Combination of Basil Oil and Taiwan Red Cypress Oil

The essential oil of Basil (Ocimum basilicum/India) and other essential oils, including Orange sweet (Citrus dulcis/Brazil), Rosemary (Rosemarinus officinalis/Spain), Cinnamon (Cinnamonum zeylanicum/Sri Lanka), Ginger (Zingiber officinale/China), Mandarin (Citrus reticulate/Argentina), Lavender (Lavendula stoechas/Spain), Eucalyptus (Eucalyptus globules/China), Tea tree (Melaleuca alternifolia/Australia), Chamomile Blue (Matricaria chamomilla/UK), Lemon balm (Melissa officinalis/India), Peppermint (Mentha piperita/USA) and Thyme (white) (Thymus vulgaris/India), were purchased from AromaGreens Workshop Co.,Ltd. (Taiwan) in 10 ml dark glass vials. The essential oil of the heartwood of Taiwan red cypress (Chamaecyparis formosensis Matsum.) and additional three essential oils, including leaves of Cajeput tree (Melaleuca leucadendron L.), Camphor tree (Cinnamomum camphora) and Basil (Ocimum basilicum), were prepared from our laboratory. The essential oils of samples were obtained by hydro-distillation for 8 h using a Clevenger-type apparatus. Ethanol (75%) and sterile water were also included for serving as controls.

As one exemplary formulation of present invention, the prepared Basil oil and Taiwan red cypress oil are combined at a ratio of 1:1 (V/V).

### Example 2

### Antibacterial Activities of the Combination of Basil Oil and Taiwan Red Cypress Oil against MRSA Strains

To investigate the application of plant essential oils against MRSA, some essential oils were chosen (including 13 commonly used in the market and 4 extracted by ourselves, as described in Example 1). Several S. aureus strains were chosen in this study, including two reference strains, a methicillin-susceptible strain, S. aureus ATCC 29213 and a MRSA strain ATCC 43300; two HA-MRSA clinical strains, V070 (ST5:SCCmec II) and M088 (ST239:SCCmec III); two CA-MRSA clinical strains, C103 (ST59:SCCmec IV) and M183 (ST59:SCCmec V)(Chen, Wang et al. 2014); and a VISA strain Z172 (ST239:SCCmec III, minimum inhibitory concentration (MIC) of vancomycin 4 μg/ml), which also exhibited daptomycin nonsusceptible phenotype (MIC >4 μg/ml). The Escherichia coli ATCC 25922 strain was used as a control.

Disk Diffusion Test

Antimicrobial susceptibility was determined by the disk diffusion test (CLSI 2015). The dried surface of a 150 mm Mueller-Hinton Agar (MHA) plate (BBL) was inoculated by streaking the inoculum suspension of MRSA strain ATCC 43300 with equivalent to that of a 0.5 McFarland standard. Sterile paper disks (6 mm in diameter) were placed into inoculated surface then loaded with 10 μl of essential oil. All plates were incubated at 35° C. for 24 h and the diameter (mm) of the zones of complete inhibition, including the diameter of the disk was measured.

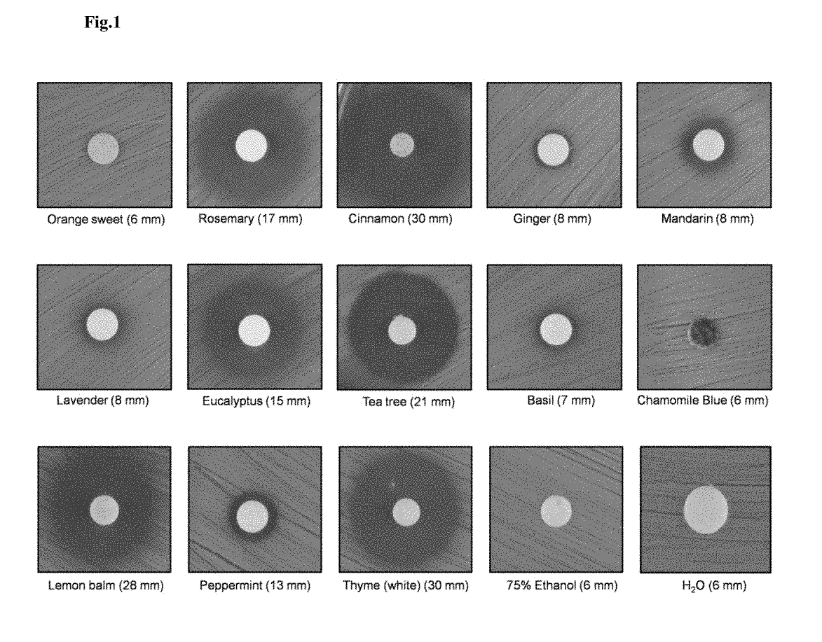

FIG. 1 shows that MRSA reference strain ATCC 43300 is susceptible to some essential oils when tested by the disk diffusion method. Six essential oils tested showed good antibacterial activity with inhibition zones had diameters ranging from 15 to 30 mm. These six essential oils were Cinnamon oil (30 mm), Thyme (white) oil (30 mm), Lemon balm oil (28 mm), Tea tree oil (21 mm), Rosemary oil (17 mm) and Eucalyptus oil (15 mm). Several studies have shown that Cinnamon, Thyme (white) and Rosemary oils had strong antibacterial activity against S. aureus. However, among the majority of effective essential oils, Tea tree oil appears to be the most studied of all essential oils in the scientific literature (Halcon and Milkus, Am J Infect Control 32(7): 402-408, 2004).

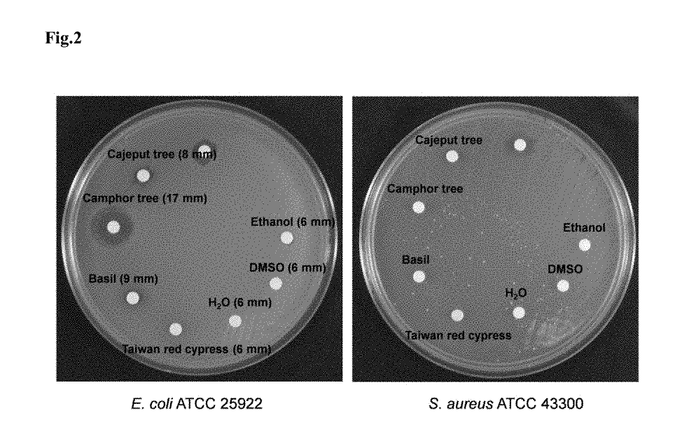

Beside the effective zones of inhibition were observed for commercial products, we were surprised to find the phenomenon of inhibition in the tested plate from our own extraction oils (FIG. 2). Comparing to the results of E. coli which only showed the moderate activity of Camphor tree oil (17 mm), most of the MRSA ATCC 43300 strain cells were killed after 24 h exposure in that plate. This phenomenon seems to be caused by the vapor activity of tested essential oils.

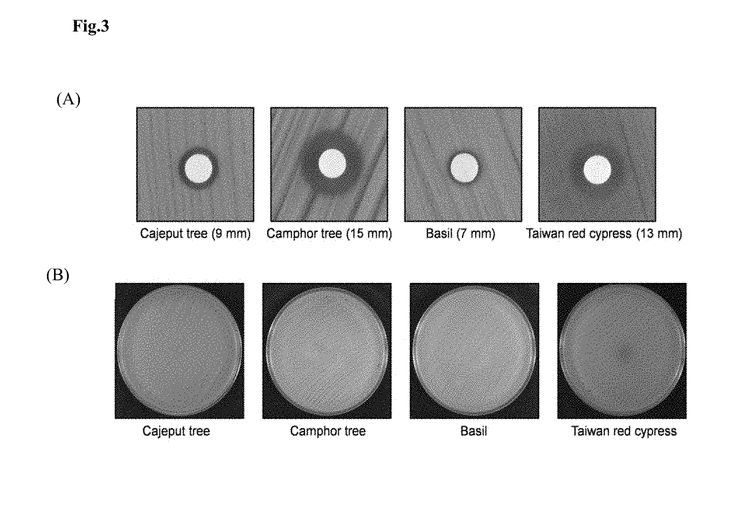

To clarify to the role of individual essential oils, disk diffusion test and vapor activity test were performed on the independent plate for each essential oil. To our surprise, among these four essential oils, only Camphor tree oil and Taiwan red cypress oil exhibited the moderate activity against ATCC 43300 strain by disk diffusion method with a zone of inhibition of 15 mm and 13 mm, respectively (FIG. 3A). However, ATCC 43300 strain showed no susceptibility to the vapors of each individual essential oil with the exception of Taiwan red cypress oil, which exhibited slight activity (FIG. 3B).

Vapor Activity Test

To explore the potential effect of combination, pair-wise combinations of these four essential oils were tested for their antibacterial activity by vapor activity test. To assay the strong vapor activity of combination in disk diffusion test, systematic evaluation of the vapor activity was using the inverted Petri dish method. Vapor activity of combination effects of oils were determined by two paper disks, each with 10 μl of essential oil. Impregnated disks were placed on the lid of the Petri dish to expose to the inverted MHA plate inoculated with test strain as disk diffusion test. All plates were incubated at 35° C. for 24 h.

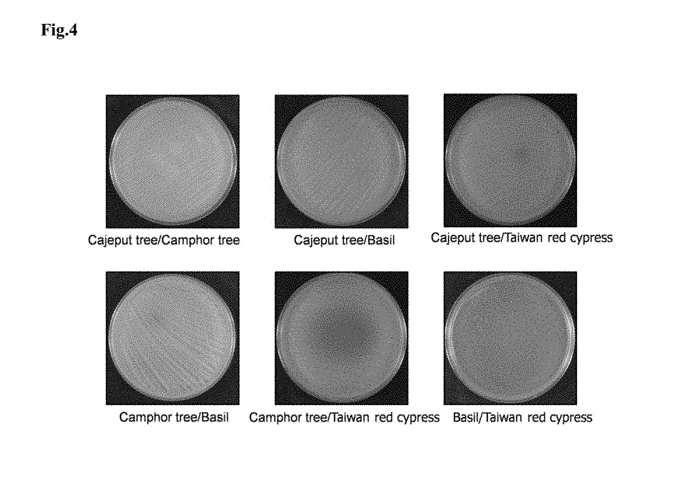

As showed in FIG. 4, four of these combinations had no effect on the enhanced activity compared to the individual oil. Conversely, the other two combinations (Camphor tree oil and Taiwan red cypress oil; Basil oil and Taiwan red cypress oil) produced enlarged zones of inhibition against ATCC 43300 strain, especially the combination of Basil oil and Taiwan red cypress oil (10 μL of each essential oil) which reproduced the inhibitory phenomenon of all four essential oil combination.

From the results of vapor activity of pair-wise combination, we assumed that Basil oil may has the ability to enhance the antibacterial activity of Taiwan red cypress oil through the vapor phase. We also found that Basil oil from commercial product and our own extraction had the same effect when combination with Taiwan red cypress oil.

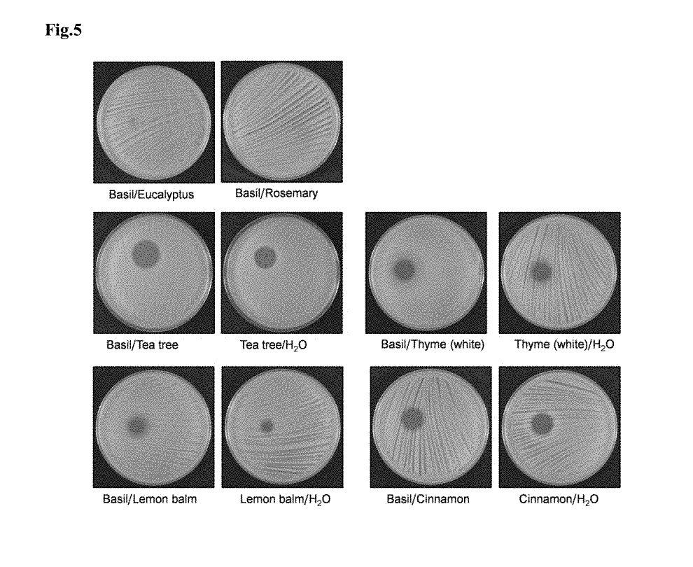

To investigate the enhancer role of Basil oil and to compare the vapor activity of six effective commercial essential oils, vapor activity test were performed on these six essential oils in combination with Basil oil or sterile water. Among these six effective essential oils, Rosemary oil and Eucalyptus oil did not show the vapor activity even in combination with Basil oil; the other four essential oils exhibited moderate vapor activity even they had strong antibacterial activity by using the disk diffusion method and only Tea tree oil and Lemon balm oil showed enhanced vapor activity when in combination with Basil oil (FIG. 5).

Several studies have shown that Cinnamon oil and Tea tree oil had the most potential bactericidal properties against various pathogens. However, our results showed that effective combination of Basil oil and Taiwan red cypress oil had most bactericidal activity against MRSA than Cinnamon oil and Tea tree oil in vapor phase. This formula has the potential to be used for the prevention of MRSA infection and to be utilized as an alternative topical decolonization agent. Tea tree oil has been widely used in phytotherapy medicine over the centuries, especially as an antiseptic agent. It also has been evaluated to compare with standard treatment by 2% mupirocin nasal ointment and triclosan body wash for topical decolonization agent against MRSA. In this study, the combination of Basil oil and Taiwan red cypress oil demonstrated promising efficacy than the Tea tree oil.

Agar Dilution Test

To confirm the efficacy of antibacterial activity of effective combination (Basil oil and Taiwan red cypress oil) on clinical isolates, individual and in combination of these two oils were tested by agar dilution assay to determine the MIC against a selection of S. aureus strains, including MSSA reference strain ATCC 29213; MRSA reference strain ATCC 43300; HA-MRSA clinical strains, V070, M088; CA-MRSA clinical strains, C103, M183; and VISA strain Z172.

MICs were determined by agar dilution method on test strains (CLSI 2015). Essential oils were prepared in the MHA by making serial twofold dilutions with Tween 80. Aliquots (1 μl) of each of the test strains were spotted onto the agar surface from an inoculum of 1:10 dilution suspension of 0.5 McFarland standard, and the MIC was read after incubation at 35° C. for 24 hr. The MIC was defined as the lowest concentration of essential oil that completely inhibits growth on the agar.

The MICs of these two oils in sole and blend against S. aureus strains are presented in Table 1. The MICs of these two oils combination showed highest activity, with a MIC of 0.5 mg/ml against all isolates except MRSA reference strain ATCC 43300. This indicated that clinical strains were more susceptible to the formula of our own blend. The combination of these two oils but not solely had significant vapor effects to inhibit the growth of multi-drug resistant MRSA indicated that these two essential oils may bind to the different targets of MRSA or cause release certain components into the vapor phase, and then caused the following death of MRSA.

TABLE 1
Minimum inhibitory concentration (MIC, mg/ml) of essential oils
exhibited effective combination against a selection of S. aureus strains.

| Strain | Basil | Taiwan red cypress | Basil/Taiwan red cypress |
|---|---|---|---|
| ATCC29213 | >4 | 1 | 0.5 |
| ATCC43300 | >4 | 2 | 1 |
| V070 | >4 | 1 | 0.5 |
| M088 | >4 | 1 | 0.5 |
| C103 | >4 | 1 | 0.5 |
| M183 | >4 | 1 | 0.5 |
| Z172 | 4 | 1 | 0.5 |

### Example 3

### Antibacterial Activities of the Combination of Basil Oil and Taiwan Red Cypress Oil against VISA Strains

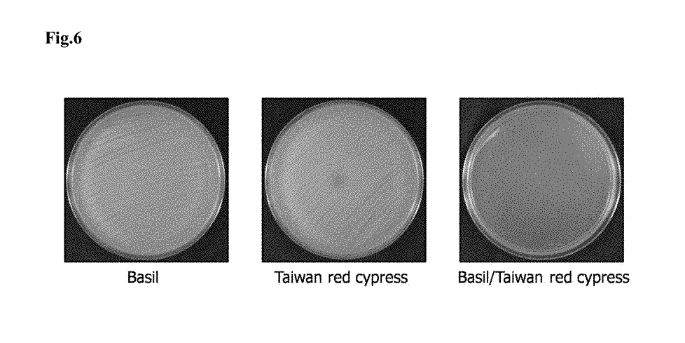

Due to the urgent need of solution to deal with the emergence of VISA strains, we also test the effect of combination of Basil oil and Taiwan red cypress oil against a daptomycin nonsusceptible VISA clinical strain Z172. Interestingly, our results showed that Z172 strain appeared to be more susceptible to the combination of Basil oil and Taiwan red cypress oil than the MRSA reference strain ATCC 43300; most of the cells were killed by the vapor activity (FIG. 6).

Based on the results described above, the use of this formula in dermatological products such as soaps, hand and body lotions, and deodorants etc. seems to be helpful for the prevention of MRSA infection. Basil oil and Taiwan red cypress oil have been used for a long time, however, there is little information regarding toxicity. Therefore, the combination of present invention may have potential to be used for medical treatment against multi-drug resistant MRSA or prophylactic eradication of S. aureus in the hospital and at home.

1. An antibacterial composition for preventing drug-resistant Staphylococcus aureus infections, comprising a Taiwan red cypress (Chamaecyparis formosensis Matsum.) essential oil combined with a Basil (Ocimum basilicum) essential oil at a ratio of 1:2 to 2:1 (V/V).

2. The antibacterial composition of claim 1, wherein the combination ratio of the Taiwan red cypress and the Basil essential oil is in a range of 1:1 (V/V).

3. The antibacterial composition of claim 1, wherein the drug-resistant Staphylococcus aureus is a Methicillin-resistant Staphylococcus aureus (MRSA) strain.

4. The antibacterial composition of claim 1, wherein the drug-resistant Staphylococcus aureus is a vancomycin intermediate (VISA) strain.

5. The antibacterial composition of claim 1, wherein the Taiwan red cypress essential oil is obtained by hydro-distillation of a heartwood of Taiwan red cypress (Chamaecyparis formosensis Matsum.).

6. The antibacterial composition of claim 1, wherein the Basil essential oil essential oil is obtained by hydro-distillation of leaves of Basil (Ocimum basilicum).

7. A method for inhibiting growth of drug-resistant Staphylococcus aureus, comprising a step of contacting the drug-resistant Staphylococcus aureus with the antibacterial composition of claim 1.

8. The method of claim 7, wherein the contacting step comprises applying the antibacterial combination to a surface where the drug-resistant Staphylococcus aureus exists.

9. The method of claim 7, wherein the contacting step comprises dispersing the antibacterial combination in an area containing the drug-resistant Staphylococcus aureus.

10. The method of claim 7, wherein the drug-resistant Staphylococcus aureus is a Methicillin-resistant Staphylococcus aureus (MRSA) strain.
